(19) United States
(12) Patent Application Publication
(10) Pub. No.: US 2026/0317294 A1
(43) Pub. Date: Oct. 15, 2026

(54) METHOD FOR DEPOLYMERIZING POLYETHYLENE TEREPHTHALATE (PET) USING REACTIVE DISTILLATION

(71) Applicants: Hyundai Motor Company, Seoul (KR); Kia Corporation, Seoul (KR); INHA University Research and Business Foundation, Incheon (KR)

(72) Inventors: Seongje Kim, Hwaseong-si (KR); Hongchan Jeon, Hwaseong-si (KR); Ha Hyun Song, Incheon (KR); Hee Joong Kim, Seoul (KR)

(73) Assignees: Hyundai Motor Company, Seoul (KR); Kia Corporation, Seoul (KR); INHA University Research and Business Foundation, Incheon (KR)

(21) Appl. No.: 19/297,656

(22) Filed: Aug. 12, 2025

(30) Foreign Application Priority Data
Apr. 15, 2025 (KR) 10-2025-0048796

(51) Int. Cl. C07D 325/00

(52) U.S. Cl. CPC C07D 325/00

(57) ABSTRACT

The present disclosure relates to a method for depolymerizing polyethylene terephthalate using reactive distillation. The method for depolymerizing polyethylene terephthalate of the present disclosure allows for efficient purification and recovery of depolymerization products, and using the method, a closed-loop recycling process improving on existing PET recycling technologies are designed.

### CROSS-REFERENCE TO RELATED APPLICATION

This present application claims the benefit of priority to Korean Patent Application No. 10-2025-0048796, filed on Apr. 15, 2025, in the Korean Intellectual Property Office, the entire disclosure of which is incorporated herein by reference in its entirety.

### FIELD

The present disclosure relates to a method for depolymerizing polyethylene terephthalate using reactive distillation.

### BACKGROUND

Polyethylene terephthalate (PET) is recognized as a commonly used thermoplastic globally, serving as a fundamental polymer in various applications, including beverage bottles, food containers, synthetic fibers, and an array of industrial processes. Its versatility and durability have led to its predominance in consumer packaging and textiles. However, the ubiquitous presence of PET in the market has also precipitated a significant surge in plastic waste, which poses profound environmental challenges. The accumulation of PET waste in the natural environment, notably in oceans and landfills, has been linked to detrimental effects on wildlife and ecosystems. As awareness of these environmental repercussions grows, the imperative for efficacious PET recycling solutions has intensified.

For example, in response to the escalating plastic waste crisis, there is an increasing impetus for the advancement of PET recycling technologies and methodologies to mitigate the environmental impact. This demand is influenced not only by consumer concern but also by the tightening of international regulations aimed at reducing plastic pollution. Governments and regulatory bodies are increasingly enacting strict guidelines and policies that foster sustainable practices and promote a circular economy model to ensure that PET products are not only recycled but also repurposed for new applications. As such, ongoing research and innovation in PET recycling processes, including chemical and mechanical recycling strategies, is at a high demand in order to address both the environmental challenges posed by plastic waste and the economic opportunities inherent in a sustainable materials economy.

Recycling technologies for PET can be fundamentally classified into two primary categories: mechanical recycling and chemical recycling. Mechanical recycling involves the physical processes of collecting, cleaning, and reprocessing PET waste into new materials, facilitating the production of recycled PET (rPET) suitable for various applications. In contrast, chemical recycling employs chemical processes to break down PET into its constituent monomers or other useful compounds, allowing for the restoration of the polymer's original quality and the potential for infinite recyclability. Ongoing research is exploring the efficiencies, challenges, and advancements associated with both mechanical and chemical recycling methods, aiming to optimize these approaches to enhance the sustainability and effectiveness of PET waste management. The matters described in this Background section are only for enhancement of understanding of the background of the disclosure, and should not be taken as acknowledgement that they correspond to prior art already known to those skilled in the art.

The matters described in this Background section are only for enhancement of understanding of the background of the disclosure, and should not be taken as acknowledgement that they correspond to prior art already known to those skilled in the art.

### SUMMARY

The present disclosure is directed to providing a method for depolymerizing polyethylene terephthalate that enables efficient purification and recovery of depolymerized products.

The present disclosure is further directed to providing a method for depolymerizing polyethylene terephthalate that can be used in a closed-loop recycling process.

The present disclosure is further directed to providing a method for depolymerizing polyethylene terephthalate that can be applied to green technology fields such as PET recycling.

An aspect of the present disclosure provides a method for depolymerizing PET, including preparing a depolymerization reaction mixture by mixing PET and ZnCl₂, obtaining a depolymerization product by subjecting the depolymerization reaction mixture to reactive distillation, and obtaining a cyclic oligomer by purifying the depolymerization product.

The preparing a depolymerization reaction mixture according to one embodiment includes mixing the PET and the ZnCl₂ in a weight ratio of 1:(0.05 to 0.4).

The preparing a depolymerization reaction mixture according to one embodiment includes further mixing bis(2-hydroxyethyl) terephthalate (BHET).

The preparing a depolymerization reaction mixture according to one embodiment includes mixing the PET, the ZnCl₂, and the BHET in a weight ratio of 1:(0.2 to 0.4):(0.05 to 0.2.).

The reactive distillation according to one embodiment is performed at a temperature of 270-350° C. and a pressure of 300 mTorr or less for 2-10 hours.

The obtaining a cyclic oligomer according to one embodiment includes purifying the depolymerization product through one or more processes selected from extraction or sublimation.

The cyclic oligomer according to one embodiment is a cyclic oligomer of ethylene terephthalate (COET).

The method for depolymerizing PET according to one embodiment further includes, before the preparing the depolymerization reaction mixture, pretreating the PET into a flake form.

Another aspect of the present disclosure provides a cyclic oligomer obtained by the method for depolymerizing PET.

Still another aspect of the present disclosure provides a method for producing recycled PET, including performing ring-opening polymerization of a cyclic oligomer.

The method for depolymerizing polyethylene terephthalate according to embodiments of the present disclosure allows for efficient purification and recovery of depolymerization products.

Additionally, using the method for depolymerizing polyethylene terephthalate according to embodiments of the present disclosure, a closed-loop recycling process is designed.

### BRIEF DESCRIPTION OF THE DRAWINGS

The foregoing and other aspects, as well as the following detailed description of the embodiments, should be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings indicate like elements.

FIG. 1 is a non-limiting flowchart showing the overall process of a method for depolymerizing PET according to one embodiment of the present disclosure.

FIG. 2 is an image showing the mechanism by which ZnCl₂ and BHET participate in PET depolymerization according to one embodiment of the present disclosure.

FIG. 3 shows ¹H-NMR analysis results of depolymerization products obtained according to an example of the present disclosure and comparative examples, to verify COET formation according to catalyst type.

FIG. 4 shows ¹H-NMR analysis results of depolymerization products obtained according to examples of the present disclosure to verify COET yield according to catalyst content.

FIG. 5 shows ¹H-NMR analysis results of depolymerization products obtained according to examples of the present disclosure to verify COET yield according to temperature.

FIG. 6 shows ¹H-NMR analysis results of depolymerization products obtained according to examples of the present disclosure to verify COET yield according to catalyst content and presence or absence of BHET.

FIG. 7A shows MALDI-TOF analysis results of synthetic COET and FIG. 7B shows MALDI-TOF analysis results of COET obtained according to an example of the present disclosure.

FIG. 8 shows ¹H-NMR analysis results of recycled PET manufactured by ring-opening polymerization of COET obtained according to an example of the present disclosure, and virgin PET.

FIG. 9 shows DSC analysis results of recycled PET manufactured by ring-opening polymerization of COET obtained according to an example of the present disclosure, and virgin PET.

FIG. 10 shows ¹H-NMR analysis results of COET obtained according to an example of the present disclosure.

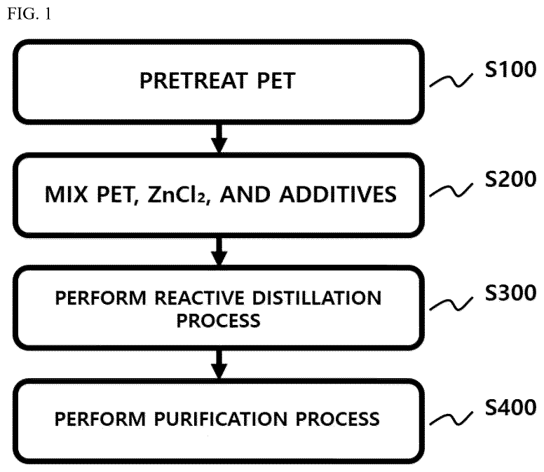

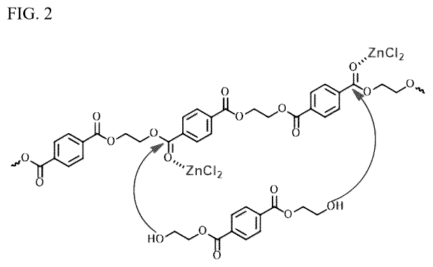

### DETAILED DESCRIPTION

Hereinafter, the present disclosure will be described in more detail. However, the following embodiments are provided merely as references for describing the present disclosure in detail, and the present disclosure is not limited thereto and may be implemented in various forms

Unless otherwise defined, all technical and scientific terms used herein have the same meaning as commonly understood by those skilled in the art to which the present disclosure pertains.

The terms used herein are intended merely to describe particular embodiments effectively and are not intended to limit the present disclosure.

Singular forms “a,” “an,” and “the” used in the specification and the appended claims are intended to include plural referents unless the context clearly dictates otherwise.

The units used in this specification, unless otherwise stated, are based on weight. For instance, the units such as “%” or “ratio” refer to weight percent (wt. %) or weight ratio, respectively. Unless otherwise defined, weight percent (wt. %) refers to the proportion of a specific component within the total composition, expressed as a percentage by weight.

When a portion is described as “including” or “comprising” a certain component, it means that, unless specifically stated to the contrary, the inclusion of other components is not excluded but rather that additional components may also be included.

In addition, numerical ranges used in this specification includes all values between the lower and upper limits, all values incrementally derived logically within shape and breadth of the defined ranges, all double-limited values, and all possible combinations of upper and lower limits of differently limited numerical ranges. Unless specifically defined in the specification of the present disclosure, values outside the defined numerical ranges that may occur due to experimental error or rounding off of values are also included within the defined numerical ranges.

The following provides a more detailed description of the present disclosure.

An aspect of the present disclosure provides a method for depolymerizing PET, including preparing a depolymerization reaction mixture by mixing PET and ZnCl₂, obtaining a depolymerization product by subjecting the depolymerization reaction mixture to reactive distillation, and obtaining a cyclic oligomer by purifying the depolymerization product.

The method for depolymerizing PET disclosed herein is efficient, as it does not require large amounts of solvent, has a high recovery rate of cyclic oligomers, and introduces reactive distillation to efficiently purify and recover depolymerization products.

In embodiments, the preparing a depolymerization reaction mixture includes preparing a depolymerization reaction mixture by mixing the PET and the ZnCl₂.

The ZnCl₂ may act as a Lewis acid, accepting lone pairs of electrons from oxygen contained in the PET, thereby lowering the energy of the transition state of the transesterification reaction. Additionally, the ZnCl₂ may increase the concentration of specific intermediates, directing the transesterification reaction toward a pathway where PET decomposes into cyclic oligomer forms.

In one embodiment of the present disclosure, in the preparing the depolymerization reaction mixture, the PET and the ZnCl₂ are mixed in a weight ratio of 1:(0.05 to 0.4), specifically 1:(0.1 to 0.3), more specifically 1:(0.15 to 0.25). When this range is satisfied, the yield of cyclic oligomers is improved.

In one embodiment of the present disclosure, the preparing a depolymerization reaction mixture includes further mixing bis(2-hydroxyethyl) terephthalate (BHET).

In embodiments, the BHET provides hydroxyl terminals for cyclic oligomer synthesis. Specifically, the BHET may play a role in inducing cyclization with hydroxyl terminals that can react with carbonyl carbon included in PET activated by ZnCl₂. Since the BHET may act as a reactive monomer, the transesterification reaction can occur more quickly when the BHET is further included in the depolymerization reaction mixture, and the selectivity of cyclic oligomers may be improved.

In one embodiment of the present disclosure, the preparing a depolymerization reaction mixture includes mixing the PET, the ZnCl₂, and the BHET in a weight ratio of 1:(0.2 to 0.4):(0.05 to 0.2), specifically 1:(0.3 to 0.4):(0.08 to 0.15). When this range is satisfied, the yield of cyclic oligomers is improved.

In embodiments, the obtaining the depolymerization product includes subjecting the depolymerization reaction mixture to reactive distillation to obtain a depolymerization product.

In one embodiment of the present disclosure, the reactive distillation is performed at a temperature of 270-350° C., specifically 290-330° C., more specifically 300-320° C. Further, the reactive distillation may be performed at a pressure of 300 mTorr or less, specifically 100 mTorr or less, with a lower limit of 104 mTorr or more. Also, the reactive distillation may be performed for 2-10 hours, specifically 4-8 hours. When these ranges are satisfied, the depolymerization efficiency and the yield of cyclic oligomers may be improved.

In embodiments, the obtaining a cyclic oligomer includes obtaining the cyclic oligomer from the depolymerization product through purification using the difference in volatility according to molecular weight, as the depolymerization product includes products such as cyclic oligomers, terephthalic acid, ethylene glycol, and the like.

In one embodiment of the present disclosure, the obtaining a cyclic oligomer includes purifying the depolymerization product through one or more processes selected from extraction or sublimation.

Specifically, the extraction is a process of converting terephthalic acid into a water-soluble salt form using a basic aqueous solution, and separating terephthalic acid from the depolymerization product by layer-separating the aqueous phase comprising terephthalic acid and the remaining organic phase. The sublimation is a process of separating depolymerization products that are not separated due to similar solubility by the difference in boiling points. Therefore, when using extraction and sublimation to purify the depolymerization product, only cyclic oligomers can be recovered, with high purity.

According to one embodiment of the present disclosure, the cyclic oligomer may be a cyclic oligomer of ethylene terephthalate (COET). The COET may be expressed as the following Chemical Formula 1.

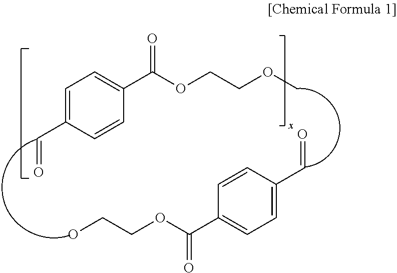

In Chemical Formula 1, x may be an integer from 1 to 5.

In one embodiment of the present disclosure, the method for depolymerizing PET further includes, before the preparing the depolymerization reaction mixture, pretreating the PET into a flake form.

In one embodiment, the pretreating the PET into a flake form includes collecting waste PET, grinding it into a flake form, washing, and drying. During grinding, the size of the flakes is not limited. During the washing, washing with a strong base is preferable. During the drying, drying at 70-100° C. for 10-14 hours is preferable. However, these specific conditions are not limited thereto as long as the objectives of the present disclosure can be achieved.

In addition, the present disclosure provides a cyclic oligomer obtained by the method for depolymerizing PET.

In addition, the present disclosure provides a method for producing recycled PET, the method including performing ring-opening polymerization of a cyclic oligomer.

The ring-opening polymerization, in embodiments, is performed using any known method, but as an example, it may be performed by adding a metal catalyst to the cyclic oligomer and heating to proceed with the ring-opening reaction, and then polymerizing and growing the ring-opened oligomer.

In one embodiment of the present disclosure, the metal catalyst is Sn(Oct)₂, a Ti-based catalyst, a Zn series catalyst, or a combination thereof.

Hereinafter, preferred examples of the present disclosure and comparative examples will be described. However, the following examples are merely preferred examples of the present disclosure, and the present disclosure is not intended to be limited thereto.

### Example 1

A depolymerization reaction mixture was prepared by mixing 10 g of waste PET ground into a flake form and 1 g of ZnCl₂. Reactive distillation was performed while stirring at 310° C., 100 mTorr for 6 hours to obtain a depolymerization product.

The depolymerization product was dissolved in dichloromethane solvent, 1 weight % sodium hydroxide aqueous solution was added, and the aqueous phase was extracted for primary purification. The primary purified depolymerization product was heated to 100° C. for secondary purification to obtain a cyclic oligomer, COET.

### Examples 2 to 4

Cyclic oligomers were obtained in the same manner as in Example 1, except that ZnCl₂ was mixed in the amounts shown in Table 1 below to prepare the depolymerization reaction mixture.

### Examples 5 and 6

Cyclic oligomers were obtained in the same manner as in Example 1, except that reactive distillation was performed at the temperature conditions listed in Table 1 below.

### Example 7

Cyclic oligomers were obtained in the same manner as in Example 1, except that 4 g of ZnCl₂ was mixed and 1 g of BHET was further mixed to prepare the depolymerization reaction mixture.

### Example 8

Cyclic oligomers were obtained in the same manner as in Example 1, except that 2 g of ZnCl₂ was mixed and 1 g of BHET was further mixed to prepare the depolymerization reaction mixture.

### Comparative Example 1

The same procedure as in Example 1 was performed, except that Yb(OTf)₃ was mixed instead of ZnCl₂ to prepare the depolymerization reaction mixture.

### Comparative Example 2

The same procedure as in Example 1 was performed, except that Sc(OTf)₃ was mixed instead of ZnCl₂ to prepare the depolymerization reaction mixture.

TABLE 1

| | PET | Catalyst (g) | | | BHET | Temperature |
|---|---|---|---|---|---|---|
| | (g) | ZnCl₂ | Yb(OTf)₃ | Sc(OTf)₃ | (g) | (° C.) |
| Example 1 | 10 | 1 | — | — | — | 310 |
| Example 2 | 10 | 2 | — | — | — | 310 |
| Example 3 | 10 | 0.5 | — | — | — | 310 |
| Example 4 | 10 | 4 | — | — | — | 310 |
| Example 5 | 10 | 1 | — | — | — | 270 |
| Example 6 | 10 | 1 | — | — | — | 285 |
| Example 7 | 10 | 4 | — | — | 1 | 310 |
| Example 8 | 10 | 2 | — | — | 1 | 310 |
| Comparative | 10 | — | 1 | — | | 310 |
| Example 1 | | | | | | |
| Comparative | 10 | 1 | — | 1 | — | 310 |
| Example 2 | | | | | | |

### Experimental Example 1: Verification of COET Formation According to Catalyst Type

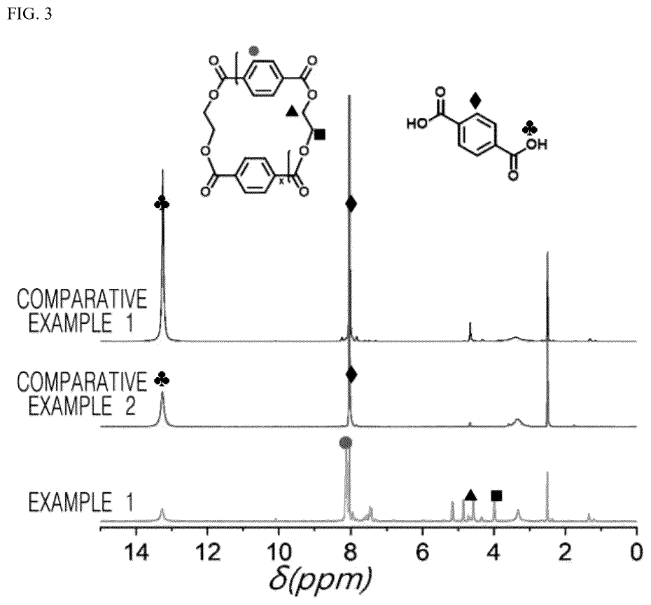

To verify COET formation according to catalyst type, depolymerization products obtained according to Example 1 and Comparative Examples 1 and 2 were analyzed through ¹H-NMR, and the results are shown in FIG. 3 and Table 2.

TABLE 2

| | COET yield |
|---|---|
| | (wt %) |
| Example 1 | 7.82 |
| Comparative | 0 |
| Example 1 | |
| Comparative | 0 |
| Example 2 | |

As can be seen in FIG. 3 and Table 2, for Comparative Examples 1 and 2, no COET peaks appeared. For Example 1, COET peaks appeared, and it was confirmed that the yield of COET calculated through ¹H-NMR was 7.82 wt %.

### Experimental Example 2: Verification of COET Yield According to Catalyst Content

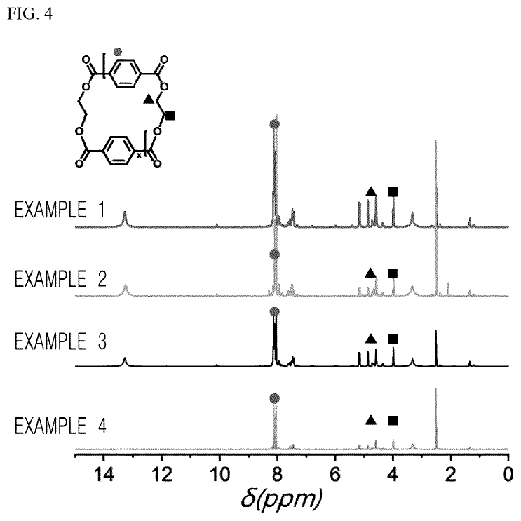

To verify COET yield according to catalyst content, depolymerization products obtained according to Examples 1 to 4 were analyzed through ¹H-NMR, and the results are shown in FIG. 4 and Table 3.

TABLE 3

| | COET yield |
|---|---|
| | (wt %) |
| Example 1 | 7.82 |
| Example 2 | 9.58 |
| Example 3 | 4.43 |
| Example 4 | 5.46 |

As can be seen in FIG. 4 and Table 3, Examples 1 and 3 had a lower COET yield compared to Example 2 due to the lower catalyst content. Example 4 had a relatively lower COET yield due to the production of byproducts such as linear oligomers, terephthalic acid, and ethylene glycol as a result of adding an excess amount of catalyst.

### Experimental Example 3: Verification of COET Yield According to Temperature

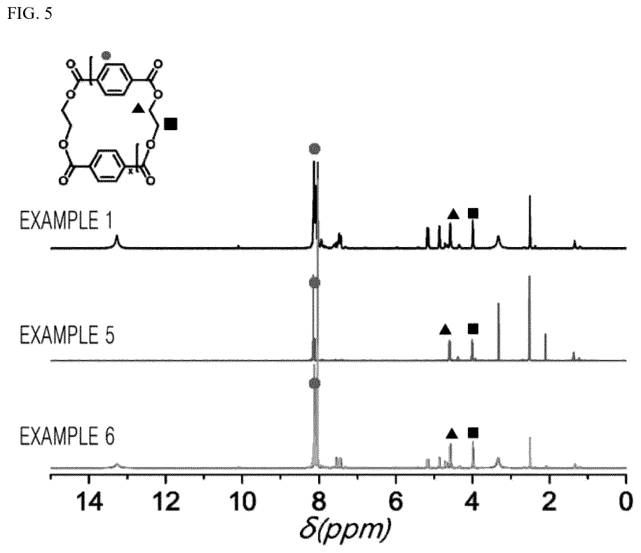

To verify COET yield according to the temperature conditions of reactive distillation, depolymerization products obtained according to Examples 1, 5, and 6 were analyzed through ¹H-NMR, and the results are shown in FIG. 5 and Table 4.

TABLE 4

| | COET yield |
|---|---|
| | (wt %) |
| Example 1 | 7.82 |
| Example 5 | 4.39 |
| Example 6 | 3.20 |

As can be seen in FIG. 5 and Table 4, for Example 1, the COET yield was improved compared to Examples 5 and 6, which is due to the faster reaction rate at higher temperatures.

### Experimental Example 4: Verification of COET Yield According to Catalyst Content and Presence or Absence of BHET

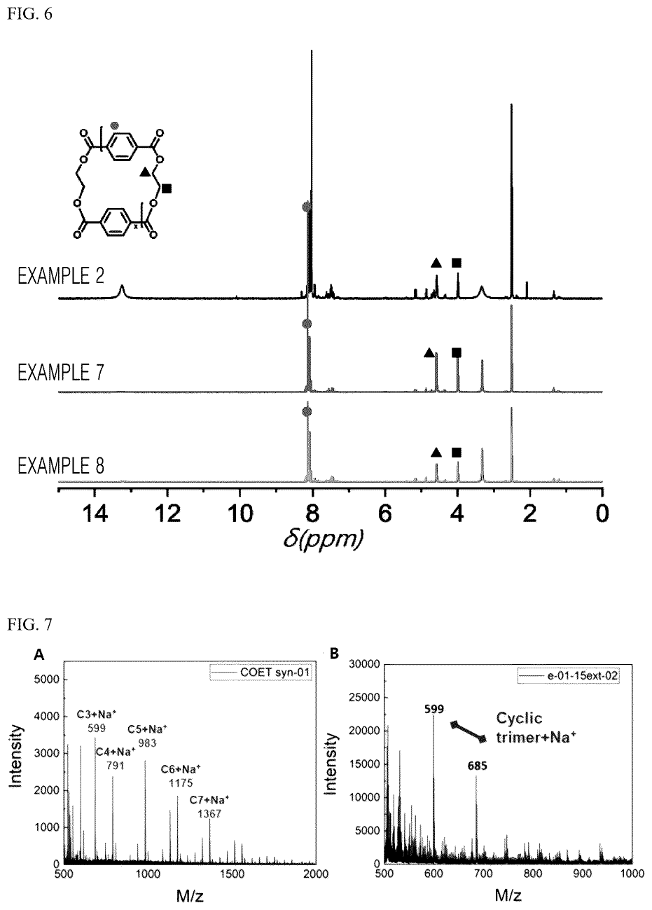

To verify COET yield according to catalyst content and presence or absence of BHET, depolymerization products obtained according to Examples 2, 7, and 8 were analyzed through ¹H-NMR, and the results are shown in FIG. 6 and Table 5.

TABLE 5

| | COET yield |
|---|---|
| | (wt %) |
| Example 2 | 9.58 |
| Example 7 | 21.4 |
| Example 8 | 12.6 |

As can be seen in FIG. 6 and Table 5, for Examples 7 and 8, which included BHET, the COET yield was improved compared to Example 2, which did not. Specifically, for Example 8, cyclization by the PET terminal and cyclization by the hydroxyl terminal of BHET occurred simultaneously, resulting in a higher COET yield compared to Example 2. For Example 7, the catalyst promoted cyclization by BHET addition, resulting in the highest COET yield.

### Experimental Example 5: COET Structure Analysis

To verify the structure of the cyclic oligomer according to the present disclosure, matrix-assisted laser desorption ionization time of flight mass spectrometry (MALDI-TOF) analysis was performed on COET obtained according to Example 1, and the results are shown in FIG. 7.

As can be seen in FIG. 7, the C3 peak (m/z=599) appearing in the synthetic COET sample was identically observed in the analysis results of COET obtained according to Example 1, confirming the presence of a trimer COET.

### Experimental Example 6: Verification of Structural and Thermal Characteristics of Recycled PET

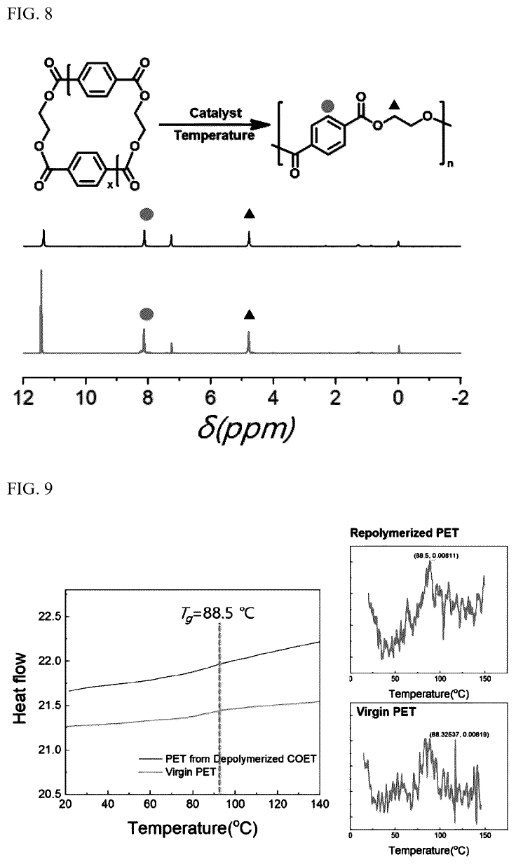

To verify the structural and thermal characteristics of recycled PET manufactured from cyclic oligomers according to the present disclosure, recycled PET was produced by adding 5 wt % of Sb₂O₃ to COET obtained according to Example 1 and performing ring-opening polymerization at 230° C. for 3 hours. The recycled PET was analyzed through ¹H-NMR and differential scanning calorimetry (DSC), and the results are shown in FIGS. 8 and 9.

As can be seen in FIG. 8, the recycled PET shows a spectrum similar to virgin PET. Also, as can be seen in FIG. 9, it was confirmed that the recycled PET and virgin PET show the glass transition temperature at the same position.

Through this, it was confirmed that recycled PET with structural and thermal characteristics similar to virgin PET can be produced.

### Experimental Example 7: Verification of COET Purity After Purification

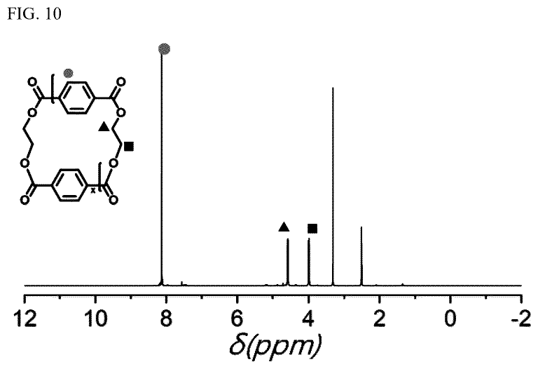

To verify the effect of purification, the cyclic oligomer obtained in Example 8 was analyzed through ¹H-NMR to measure the purity of the cyclic oligomer, and the results are shown in FIG. 10.

As can be seen in FIG. 10, in the case of Example 8, where the depolymerization product underwent extraction and sublimation purification, it was confirmed that COET showed a purity of over 99%.

Through the above experimental examples, it was confirmed that PET can be depolymerized into COET with high yield using the method for depolymerizing PET of the present disclosure. It was also confirmed that recycled PET with structural and thermal characteristics similar to virgin PET can be produced by performing ring-opening polymerization of the COET obtained through this method.

The method for depolymerizing PET of the present disclosure does not require prior sorting of PET of the same kind from plastic waste during depolymerization, and during repolymerization, byproducts such as ethylene glycol are not produced, saving the energy needed to remove them. Also, in the case of condensation polymerization, there is a limit to polymerizing to a high molecular weight as polymer terminals attach to each other, whereas in the case of ring-opening polymerization, monomers attach to the terminal, enabling the achievement of a relatively high molecular weight.

Therefore, it can be seen that an efficient PET recycling process can be designed using the method for depolymerizing PET of the present disclosure.

The features, structures, effects, and the like described in the exemplary embodiments above are included in at least one embodiment of the present disclosure and are not necessarily limited to a single embodiment. Furthermore, the features, structures, effects, and the like exemplified in each exemplary embodiment can be combined or modified in other embodiments by those skilled in the art to which the embodiments pertain. Therefore, such combinations and modifications should be construed as being within the scope of the present disclosure.

What is claimed is:

1. A method for depolymerizing polyethylene terephthalate (PET), comprising:
preparing a depolymerization reaction mixture by mixing PET and ZnCl₂;
obtaining a depolymerization product by subjecting the depolymerization reaction mixture to reactive distillation; and
obtaining a cyclic oligomer by purifying the depolymerization product.

2. The method of claim 1, wherein the preparing a depolymerization reaction mixture comprises mixing the PET and the ZnCl₂ in a weight ratio of 1:(0.05 to 0.4).

3. The method of claim 1, wherein the preparing a depolymerization reaction mixture comprises further mixing bis(2-hydroxyethyl) terephthalate (BHET).

4. The method of claim 3, wherein the preparing a depolymerization reaction mixture comprises mixing the PET, the ZnCl₂, and the BUET in a weight ratio of 1:(0.2 to 0.4):(0.05 to 0.2).

5. The method of claim 1, wherein the reactive distillation is performed at a temperature of 270-350° C. and a pressure of 300 mTorr or less for 2-10 hours.

6. The method of claim 1, wherein the obtaining a cyclic oligomer comprises purifying the depolymerization product through one or more processes selected from extraction or sublimation.

7. The method of claim 1, wherein the cyclic oligomer is a cyclic oligomer of ethylene terephthalate (COET).

8. The method of claim 1, further comprising, before the preparing the depolymerization reaction mixture, pretreating the PET into a flake form.

9. A cyclic oligomer obtained by the method according to claim 1.

10. A method for producing recycled PET, comprising performing ring-opening polymerization of the cyclic oligomer of claim 9.
